(19) United States
(12) Patent Application Publication
(10) Pub. No.: US 2003/0181356 A1
(43) Pub. Date: Sep. 25, 2003

(54) Compositions and methods for treating emphysema

(76) Inventor: Edward Ingenito, Kingston, MA (US)

Correspondence Address: FISH & RICHARDSON PC, 225 FRANKLIN ST, BOSTON, MA, 02110, US

(21) Appl. No.: 10/386,149

(22) Filed: Mar. 11, 2003

Related U.S. Application Data
(60) Provisional application No. 60/363,118, filed on Mar. 11, 2002.

(51) Int. Cl. A61K 38/17; A61K 31/685; A61K 31/56; A61K 31/202

(52) U.S. Cl. 514/2; 514/78; 514/171; 514/560

(57) ABSTRACT

The present invention features compositions and methods for treating emphysema by reducing the amount of force the fibers in the lung (e.g., the collagen and elastin fibers in the walls of the alveoli) must bear. More particularly, in one embodiment, the invention features a pharmaceutically acceptable composition comprising a lipid that, when applied to an enlarged alveolus (e.g., an alveolus having a diameter substantially larger than (e.g., 5, 10, 20, 50, or 100% or more than) the average alveoli in a healthy patient (i.e., a patient with no discernable lung disease), exerts a surface tension within the alveolus that substantially reduces the stress on fibers within the alveolus when inflated by a normal inspiration. The composition can display a γ* of about 30 to about 70 dynes/cm.

### RELATED APPLICATIONS

This application claims the benefit of the filing date of U.S. Ser. No. 60/363,118, which was filed on Mar. 11, 2002. The contents of the prior provisional application is hereby incorporated by reference in its entirety.

### TECHNICAL FIELD

This invention features compositions and methods for treating patients who have certain lung diseases, such as emphysema.

### BACKGROUND

Emphysema, together with asthma and chronic bronchitis, represent a disease complex known as chronic obstructive pulmonary disease (COPD). These three diseases are related in that they each cause difficulty breathing and, in most instances, they progress over time. There are substantial differences, however, in their etiology, pathology, and prognosis. For example, while asthma and chronic bronchitis are diseases of the airways, emphysema is associated with irreversible, destructive changes in lung parenchyma distal to the terminal bronchioles. Cigarette smoking is the primary cause of emphysema; the smoke triggers an inflammatory response within the lung, which is associated with an activation of both elastase and matrix metallo-proteinases (MMPs). These enzymes degrade key proteins that make up the tissue network of the lungs (Shapiro et al., Am. J. Resp. Crit. Care Med. 160:s29-s32, 1999; Hautamaki et al., Science 277:2002-2004). In fact, the pathological determinant of lung dysfunction in emphysema seems to be the progressive destruction of elastic tissue, which causes loss of lung recoil and progressive hyper-expansion.

Almost two million Americans and at least three times that many individuals worldwide suffer from emphysema (American Thoracic Society, Am. J. Resp. Crit. Care Med. 152:s77-s121, 1995). The average patient with emphysema reaches a critical level of compromise by about the age of 60 and, at that point, often begins to experience symptoms such as shortness of breath. In addition, functional capacity becomes reduced, quality of life is compromised, and the frequency of hospitalization is increased. Despite aggressive public health initiatives, cigarette smoking remains common, and emphysema will likely remain a major public health problem well into the new millennium.

Even though emphysema is a distinct condition, the therapies that have been developed to treat it are patterned after those used to treat asthma and chronic bronchitis. The treatments can be grouped into five categories: (1) inhaled and oral medications that help open narrowed or constricted airways by promoting airway muscle relaxation; (2) inhaled and oral medications that reduce airway inflammation and secretions; (3) oxygen therapy, which is designed to delay or prevent the development of pulmonary hypertension and cor pulmonale (right ventricular failure) in patients with chronic hypoxemia; (4) exercise programs that improve cardiovascular function, functional capacity, and quality of life; and (5) smoking cessation programs to delay the loss of lung function by preventing progression of smoking-related damage (Camilli et al., Am. Rev. Resp. Dis. 135:794-799, 1987). Although each of these approaches has been shown to have beneficial effects in this patient population, only oxygen therapy and smoking cessation significantly alter the natural history of this disease (Nocturnal Oxygen Therapy Trial Group, Ann. Intern. Med. 93:391, 1980).

### SUMMARY

As noted above, in certain pulmonary diseases such as emphysema, the fiber network within the lung is progressively destroyed. As a result, recoil pressures within the lung decrease and, over time, each remaining fiber must support more and more force. At some point, fibers are stressed to the point where they break due to the strain of normal breathing. The notion that stress-related fiber rupture contributes to the progression of emphysema represents a shift from conventional thinking.

The present invention features compositions and methods for treating emphysema by reducing the amount of force the fibers in the lung (e.g., the collagen and elastin fibers in the walls of the alveoli) must bear. The compositions of the invention may be referred to herein as “surface films” because they are applied to the inner surface of alveoli, typically through the bronchial tree (the alveoli are very small, sac-like structures at the terminal portions of the bronchial tree; oxygen and carbon dioxide are exchanged with the blood where capillaries contact the alveoli). The films are defined not only by their composition per se, but also by virtue of the biophysical properties they display. The content of the present surface films, and the biophysical properties that result, are distinct from those of either normal surfactant or the surfactant replacements presently known in the art (e.g., EXOSURF and SURVANTA; e.g., EXOSURF does not have a minimum surface tension of <5 dynes/cm). The replacements presently known are used to treat diseases in which surfactant dysfunction is the primary abnormality (e.g., acute respiratory distress syndrome (ARDS), infant hyaline membrane disease, and congenital diaphragmatic herniation). Accordingly, they strive to mimic normal surfactant. As a consequence, replacement surfactants are ineffective in treating emphysema, where there is little or no surfactant dysfunction.

The Examples below describe systematic analyses of the biophysical properties of a wide range of lipid-based surface films that provide k₁, k₂, γₘᵢₙ, and m₂ values (these parameters are defined below (γ may also appear herein as “g”) that may be similar to those of naturally occurring surfactants but, unlike natural or replacement surfactants, have γ* values greater than about 30 dynes/cm (e.g., greater than about 32, 35, 40, 45, 50, 55, 60, 65 or 70 dynes/cm). When a surface active material (like a surfactant) is added to a solution, it preferentially partitions at the air-liquid interface because that position is thermodynamically favorable. The surface tension that exists at the air-liquid interface (γ) is a function of two factors: (1) the specific surfactant added; and (2) the amount of surfactant added. When only a small amount of surfactant is added, the surface tension drops slightly. When more surfactant is added, the surface tension drops further. As more and more surfactant is added, however, a limit is reached at which addition of further surfactant does not further lower the surface tension. This limit is γ*. Unlike γ, which is a function of both surfactant concentration and surfactant type, γ* is only a function of the type of surfactant. It is the surface tension achieved in the limit that the concentration goes to infinity. γ* is an intrinsic quality of a surfactant, surface film, or any other surface active material.

In one embodiment, the invention features pharmaceutically acceptable compositions comprising a lipid (and, in alternative embodiments, further comprising a protein (or peptide) and/or a polysaccharide). While lipids have been included in other compositions applied to the lungs, the lipid components of the surface films described here are different from those previously applied. Here, when surface films possessing critical biophysical characteristics are applied to an enlarged alveolus (e.g., an alveolus having a diameter greater than about 200-300μ), they exert a surface tension within the alveolus that reduces the stress on fibers within the alveolus when it is inflated by a normal inspiration or, more preferably, a normal, deep inspiration. The stress reduction should be sufficient to inhibit fiber rupture (i.e., to reduce the number of fibers that break or to prolong the time period over which they break, relative to that observed in the lung of an untreated patient or the lung of a patient treated with a presently known surfactatant, such as EXOSURF). While stress reduction can be assessed on a physiological level (e.g., fiber rupture), it can also be assessed by an improvement in any other objective or subjective measure of a patient's overall health or pulmonary status. Thus, a lipid-based composition having one or more of the features described herein (e.g., a γ* as described herein) exerts a surface tension within an enlarged alveolus (or a population of alveoli having an average diameter greater than those of the alveoli in a healthy person or other animal) that substantially reduces the stress on fibers within the alveolus when inflated by a normal inspiration. As noted below, the enlarged alveolus may be in a patient who has a pulmonary disease, such as emphysema, and the stress reduction can be evident by an examination of the lung, of the fibers therein, or by an external parameter such as an improvement in the patient's health (e.g., an improvement in the ease of breathing or improvement in the ability to exert oneself; a slowing of the disease progression is also an indication that the surface film has reduced surface tension).

Based on clinical observations among patients with advanced emphysema who have undergone lung volume reduction therapy, and on recent experimental observations, it appears that fibers in the lungs of patients with emphysema can rupture at inflation pressures of 10-20 cm H₂O. To prevent rupture, surface films should ideally support 50-75% of the recoil that occurs when a patient takes a deep breath. For alveoli of about 300μ in diameter, the surface tension generated by such a film would have to reach about 50 dynes/cm. As described further below, the composition of the surface film can vary, so long as the film displays a surface tension-surface area profile in which surface tensions are large enough at the end of an inspiration to substantially reduce the stress on fibers with the alveolus and, at the same time, small enough at the end of expiration to substantially prevent alveolar collapse (otherwise, the surface films would adversely affect gas exchange). A film substantially reduces the stress on the fibers when it reduces the stress to the point where the patient can expect, or does experience, either an improvement in their condition or a reduction in the pace at which the disease process has occurred.

Although increasing the surface tension on the surface of alveoli, to any extent, tends to reduce the stress imparted to the fiber network, administration of an agent that produces high surface tensions uniformly throughout the lung can have dangerous consequences.

The surface films of the present invention will benefit patients, particularly those with emphysema, as there is presently no therapy that slows the progression of this disease. Even patients who undergo a volume reduction procedure will benefit, as function declines in this patient group at an accelerated rate following short-term improvement. The patients may have undergone a surgical lung volume reduction (as described in Cooper et al., J. Thorac. & Cardiovasc. Surg. 112:1319-1330, 1996) or a non-surgical reduction (as described in Ingenito et al., Am. J. Respir. Crit. Care Med. 164:295-301, 2001).

Furthermore, the compositions and methods described herein can provide benefits similar to LVRS without the associated surgical risk. (The present compositions and methods can be used in lieu of, as well as in addition to, LVRS). Because the recoil force generated by a surface film varies with the size of the surface area upon which it is spread, large alveoli, which undergo small area excursions during respiration, experience larger inward recoil forces than smaller alveoli. As a result, the surface films described here can actually shrink large, dysfunctional alveoli, and improve lung function by producing the equivalent of a chemical, surface-film-induced volume reduction. Surface films that slow the progression of emphysema will be safer and more effective if they are not toxic (following either acute or chronic administration) and have little or no impact on the synthesis or turnover or normal surfactant. As described further herein, the surface tension-surface area profile is important, and the profile of a surface film should be such that surface tensions are larger at large lung volumes (end inspiration), when stress on the fiber network is greatest, and lower at low lung volumes (end expiration) so as not to cause alveolar collapse. Optimal surface films should function well over surface area excursions equivalent to those that occur during tidal breathing as well as more labored breathing. In addition, they should, optimally, produce beneficial effects that last at least several hours (otherwise dosing schedules can be inconvenient). As the surface films of the present invention are not extracts of a naturally occurring surfactant, it is highly unlikely they will contain viral or proteinaceous contaminants, such as prions. The link between bovine spongiform encephalopathy (BSE) and human Creutzfeldt-Jakob disease is a reminder of the risk a patient must bear when they are treated with an animal product. Given that the surface films of the invention contain lipids, it is expected that they will be relatively inexpensive to manufacture and, therefore, readily available to all.

More particularly, in one embodiment, the invention features a pharmaceutically acceptable composition comprising a lipid that, when applied to an enlarged alveolus (e.g., an alveolus having a diameter substantially larger than (e.g., 5, 10, 20, 50, or 100% or more than) the average alveoli in a healthy patient (i.e., a patient with no discernable lung disease), exerts a surface tension within the alveolus that substantially reduces the stress on fibers within the alveolus when inflated by a normal inspiration. To be therapeutically effective, the composition must reduce the stress on fibers within the alveolus to the point where the fibers do not break or break at a lower rate than they would break in the absence of the composition (i.e., in an untreated patient or a patient treated with a known surfactant). The therapeutic effectiveness can be determined by following the course of the patient's disease (effectiveness being exhibited as a decline in disease progression) or by assessing objective signs or clinical symptoms of the disease (effectiveness being exhibited as an improvement in one or more of these signs or symptoms). As noted above, the composition can display a surface tension-surface area profile in which surface tensions are large enough at the end of an inspiration to substantially reduce the stress on fibers within the alveolus and, in addition, small enough at the end of an expiration to substantially prevent alveolar collapse (e.g., a profile substantially similar to that shown in FIG. 6). The composition can display a γ* of about 30 to about 70 dynes/cm (e.g., about 35 to about 65 dynes/cm; about 40 to about 60 dynes/cm; about 45 to about 55 dynes/cm; or a γ* of at least 32, 35, 40, 45, 50, 55, 60, 65, or 70 dynes/cm).

The lipid can be, for example, di-arachidonyl-phosphatidylcholine (DAPC; e.g., at least about 50% DAPC (e.g., 50, 55, 60, 65, 70, 75, or 80% DAPC), and the composition can further include di-palymitoylphosphatidylcholine (DPPC; e.g., 5-30% DPPC (e.g., 5-25%, 5-15%, 5-10% or 6, 7, 8, 9, 12, 15, 18, 20, or 25% DPPC)). Compositions with one or both of these lipids can further include phosphatidylglycerol, arachidic acid, palmitic acid, cholesterol, and/or one or more proteins or peptides (e.g., natural surfactant protein B, natural surfactant protein A, natural surfactant protein C, recombinant surfactant protein C, small alpha-helical peptides with hydrophobic characteristics, or other peptide-like compounds). In a particular embodiment, the composition can include, for example, 50-80% di-arachidoylphosphatidylcholine (DAPC), 10-30% phosphatidylglycerol, 1-10% palmitic acid, and 1-10% arachidic acid, selected so the total lipid composition does not exceed 100% of the composition. In addition, any of the lipid-based surface films of the invention can also include an anti-inflammatory agent, a steroid (e.g., hydrocortisone, dexamethasone, beclamethasone, or fluticasone), a bronchodilator, an anti-cholinergic compound, or an agent that modulates inflammation or airway tone. The compositions of the invention can also include a marker (e.g., a fluorochemical) to allow for the detection of the composition in the target area.

The compositions of the invention can be used to treat a patient (e.g., a human patient) who has emphysema or any other pulmonary disease in which fibers within the alveoli are under increased stress. The patient may have undergone a surgical or non-surgical lung volume reduction therapy.

Given its utility in treating patients with emphysema, the composition can be formulated for administration by inhalation, or by instillation of the surface film into the lung through the trachea. Thus the invention features the surface film compositions described herein formulated for administration by inhalation (e.g., as a dry powder) or instillation (e.g., as a liquid solution in water or buffered physiological solutions (e.g., saline)).

The invention also features devices comprising the surface film compositions described herein. In one embodiment, the invention includes a portable inhaler device suitable for dry powder inhalation including the surface film compositions described herein. Many such devices, typically designed to deliver anti-asthmatic agents (e.g., bronchodilators and steroids) or anti-inflammatory agents into the respiratory system are commercially available. The device can be a dry powder inhaler, which can be designed to protect the powder from moisture and to minimize any risk from occasional large doses. The inhaler can be a single-dose inhaler or a multi-dose inhaler. In another embodiment, the invention includes a nebulizer, for example, an ultrasonic nebulizer or a pressure mesh nebulizer, comprising the surface films of the invention.

The invention also features kits that, in addition to the surface film, contain, for example, a vial of sterile water or a physiologically acceptable buffer. Optionally, the kit can contain an atomizer system to generate particulate matter (atomizers are presently commercially available) and instructions for use and other printed material describing, for example, possible side effects.

The contents of all references, published patent applications and patents cited throughout the present application are hereby incorporated by reference in their entirety. The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is a schematic representation of the alveolar compartment and the forces balanced within it.

FIG. 2 is a pair of fluorescent microscopy images of a collagen fiber network in the lung before (top) and after (bottom) forty percent strain amplitude. The alveolar wall is labeled. An intact hexagonal network is evident before the tissue is stretched. Following stretch, the network is incomplete, demonstrating fiber rupture (Kononov et al., Am. J. Resp. Crit. Care Med. 164:1920-1926, 2001).

FIG. 3 is an image generated from a finite element computer model simulation. It illustrates stress distribution in a system analogous to an emphysema lung with pre-existing bullous regions, or holes. The highest stress is at the edges of these regions, where fiber rupture leads to enlarged bullae, persistent localized concentrations of stress, and additional fiber failure Suki et al. Am. J. Resp. Crit. Care Med. 163:A824, 2001).

FIG. 4 is a graph comparing surface tension (dynes/cm) to the surface area profile for normal surfactant at a concentration of 1 mg/ml. Minimum surface tension is less than 1 dyne/cm, which minimizes the tendency for alveolar collapse at low volumes. At full inflation, normal surfactant exerts a surface tension of about 30 dynes/cm.

FIG. 5 is a graph depicting the ability of a surface film to fully support distending pressures at different alveolar radii. Films that can exert higher surface tension can support significantly more distending pressures.

FIG. 6 is a graph depicting the biophysical properties of a surface film that one would expect to be effective in treating a patient with emphysema. The film has a high γₘₐₓ and low γₘᵢₙ, which would allow it to support distending pressures near full lung inflation without promoting collapse near end expiration.

FIG. 7 is a graph generated by a computer model. The graph plots surface tension (γ(dynes/cm)) against area (mm²), describing the distinct states of surface film behavior as surface area changes during cyclic oscillations simulating breathing.

FIG. 8 is a graph of an isotherm for native calf lung surfactant. The isotherm represents the relationship between the concentration of surfactant in the solution (here, expressed as the concentration of surfactant relative to the amount required to reach γ*, equal to G/G*) and surface tension γ. The open circles represent data recorded for calf lung surfactant at different concentrations expressing this relationship under equilibrium conditions. The open triangles represent data recorded for calf lung surfactant under quasi-static conditions during slow compression from equilibrium.

FIG. 9 is a pair of graphs showing surface tension, surface area profiles measured for normal calf lung surfactant (left-hand graph) and a corresponding matching computer simulation (right-hand graph) using the following parameter set: K₁=6×10⁵ ml/g/min; K₂=5 ml/g; γ*=22.2 dynes/cm; γₘᵢₙ<0.5 dynes/cm, and slope B to D, designated M₂=170 dynes/cm.

FIG. 10 is a graph depicting surface tension (dynes/cm) versus surface area (mm²) for films having different equilibrium surface tensions (γ*).

FIG. 11 is a graph showing surface tension-surface area profiles for a mixture of di-arachidonylphosphatidylcholine (PC), phosphatidylglycerol (PG), palmitic acid (PA), and arachidic acid (AA) (for a dA/A of 75%). The profiles, measured by pulsating surfactometry, are shown at 1, 20, and 100 cycles/minute. The behavior is described by k₁, k₂, γ*, and m₂ values listed in Table 1.

FIG. 12 is a graph summarizing airway resistance (Raw) in C57BL/6 mice and Tsk (+/−) mice at baseline, and at two, 10, 20, and 60 minutes following treatment with either saline or a lipid-based composition of the invention (i.e., a composition containing 70% DAPC, 20% phosphatidylglycerol, 5% DPPC and 5% arachidonic acid).

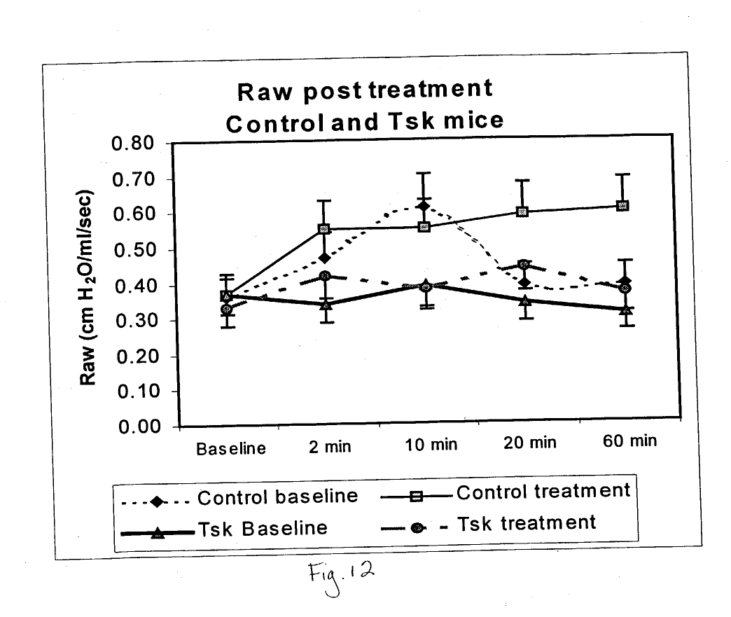

FIG. 13 is a graph summarizing tissue resistance (G) in C57BL/6 mice and Tsk (+/−) mice at baseline and at two, 10, 20, and 60 minutes following treatment with either saline or a lipid-based composition of the invention (i.e., a composition containing 70% DAPC, 20% phosphatidylglycerol, 5% DPPC and 5% arachidonic acid).

FIG. 14 is a graph summarizing quasi-static deflation pressure volume curves for C57BL/6 mice and Tsk (+/−) mice. Volumes at 0 Ptp were measured by water immersion volume displacement. The P-V relationships for Tsk mice are shifted up and to the left, consistent with the physiology of emphysema. Volumes at 0 Ptp are increased in Tsk mice, consistent with an increase in trapped gas compared to control.

FIG. 15 is a pair of graphs summarizing quasi-static pressure volume curves for control C57B/6 mice (left-hand graph) and Tsk (+/−) mice (right-hand graph) following either saline administration (solid line) or treatment with a lipid-based composition of the present invention (dashed line). In the treated mice, there is a significant rightward shift in the curves for both strains of mice, indicating increased recoil. Surfactant caused a greater reduction in trapped gas in Tsk (+/−) mice than in control.

### DETAILED DESCRIPTION

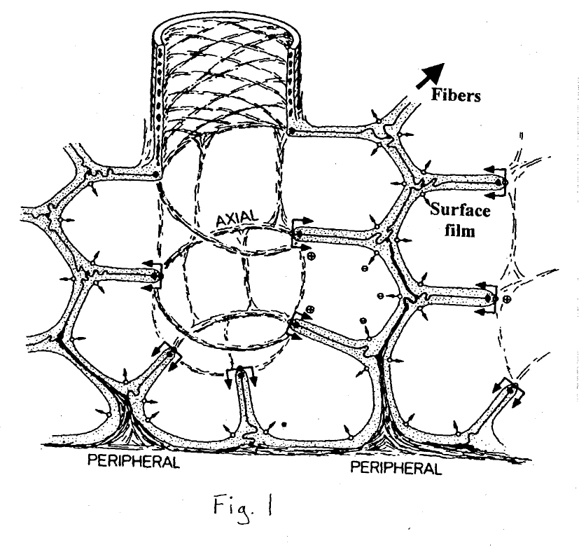

The compositions described herein were designed in, and have been tested in, the context of lung disease (more specifically, emphysema; see the tissue-based, computer-based, and in vivo models in the Examples). These models can be used to assess several parameters important for lung function, including recoil pressure and other biophysical properties of surface films and surfactants. In the lung, recoil pressures are determined by two factors: the recoil pressure that results from stretching the tissue fiber network and the recoil pressure that results from surface tension generated by the surfactant that is present at the surface of the alveoli (i.e., at the air-liquid interface). These pressures are illustrated in FIG. 1, where forces transmitted along the alveolar septae are borne by the fibers (large arrows), while inward recoil is imposed by the surface film and is distributed within the individual alveoli (small arrows).

At equilibrium (e.g., during a breath-hold following a deep inhalation), the force balance within the lung can be described by the following relationship:

P_{distending}=Pₜᵢₛₛᵤₑ+P_{surface tension}

where P_{distending} is the distending pressure in the lung generated by the enclosed gas volume, Pₜᵢₛₛᵤₑ is the recoil pressure generated by the fiber network, and P_{surface tension} is the surface tension pressure generated by the surfactant lining the alveoli (Stamenovic, Physiol. Rev. 70:1117-1134, 1990). Distending pressures are greatest at the end of an inspiration, or following a deep breath, when the lung is inflated. At these points in the respiratory cycle, Pₜᵢₛₛᵤₑ is most likely to exceed the fiber yield limit, leading to rupture.

The surface films described herein influence the force balance within the lung. While the films are not limited to any that function by a particular mechanism, we believe the films can influence the force balance, not by changing Pₜᵢₛₛᵤₑ, the major determinant of lung dysfunction in emphysema, but by altering P_{surface tension}. Thus, and without confining the invention to compositions that work by a particular mechanism, the surface films described here are thought to affect the equilibrium relationship described by the equation above by increasing P_{surface tensions}, which, in turn, relieves stress on the fibers that act mechanically, and in concert with Pₜᵢₛₛᵤₑ, to support the distending forces within the lung. Relieving that stress increases recoil pressures near total lung capacity and improves lung function in patients whose tissue recoil is decreased (e.g., patients with emphysema). By protecting the fiber network within the lung, disease progression is slowed. Furthermore, increasing P_{surface tension} (and improving tissue recoil) prolongs the benefits of lung volume reduction.

Surgical therapy has recently been introduced as an adjunct to the medical treatments described above, and the results have been impressive. The surgical approach, known as lung volume reduction surgery (LVRS), improves lung function, exercise capacity, breathing symptoms, and quality of life in the majority of emphysema patients who meet designated selection criteria (Cooper et al., J. Thorac. Cardiovasc. Surg. 109:106-116, 1995). In LVRS, damaged, hyper-inflated lung is removed, which allows a better fit between the over-expanded lung and the more normal sized chest wall. The fraction of the lung that remains within the chest cavity can better expand, and this increases the proportion of lung that can effectively contribute to ventilation (Fessler et al., Am. J. Resp. Crit. Care Med. 157:715-722, 1998). Recoil pressures increase, and expiratory flows improve. To date, LVRS is the only treatment that directly addresses lung hyper-expansion, which is the primary physiological abnormality of emphysema.

Unfortunately, in some cases the benefits of LVRS may decline over time. Peak responses occur a year or so following surgery, but they can diminish thereafter. Within three to four years, many LVRS patients may have returned to a pre-treatment functional status despite large initial improvements (Gelb et al., Am. J. Resp. Crit. Care Med. 163:1562-1566, 2001).

Accordingly, the compositions of the invention can be administered to a patient who has a pulmonary disease in which the fiber network within the alveoli is compromised (i.e., more susceptible to rupture than in patients without lung disease). Such patients include those with emphysema, and patients who have emphysema can be treated before or after any lung volume reduction (whether made by surgical or non-surgical techniques). For example, the compositions and methods of the present invention can be used in conjunction with those described in WO 01/13908.

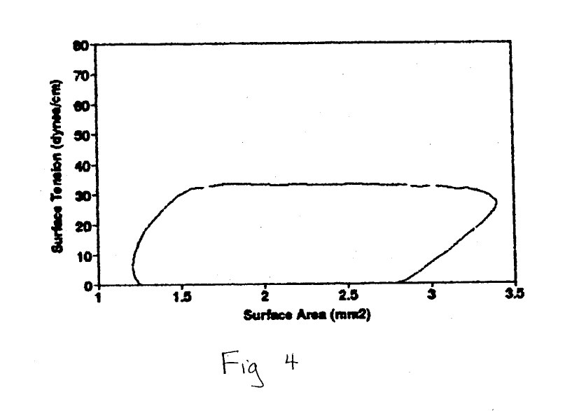

The biophysical properties of surface films. FIG. 4 illustrates the surface tension-surface area behavior of naturally occurring lung surfactant. Minimum surface tension is less than about 0.5 dynes/cm and maximum surface tension is about 32 dynes/cm. The distending pressure that can be supported by this surfactant at maximal expansion is a function of the regional alveolar radius, as expressed through Laplace's law:

ΔP=2γ/r

where ΔP is the distending pressure across the alveolus, γ is the film surface tension, and r is the alveolar radius. For a normal alveolus, which has a radius of about 100 microns, the surface film can support distending pressures of about 6.3 cm H₂O. The fiber network must support distending pressures above that. In pulmonary diseases where the fiber network is damaged or progressively destroyed, and the mean alveolar size increases, the ability of the surface film to support distending pressures decreases. For example, for an alveolus that has increased to about 300 μm in diameter, normal surfactant can support a distending pressure of only 2.1 cm H₂O. Therefore, if the distending pressure at total lung volume following a deep breath is 10 cm H₂O (a typical value for a patient with severe emphysema) and the yield stress of the fibers in the alveolar wall is about 7.0 cm H₂O, a natural surfactant can protect fibers in alveoli that are about 100 μm in diameter, but not in those having diameters of about 300 μm.

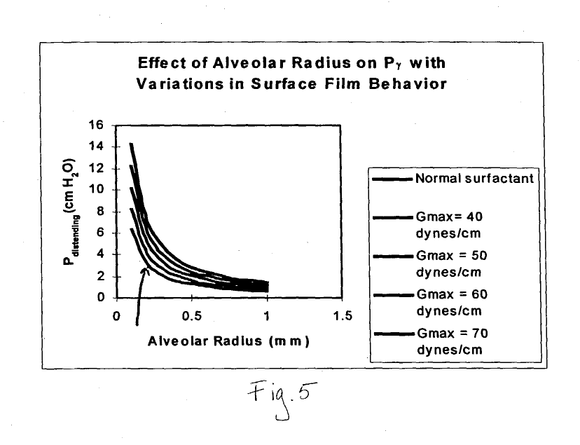

FIG. 5 shows the range of distending pressures that can be supported by surface films lining alveoli of different sizes. Each line represents a film with a different maximal surface tension, ranging from normal with a γₘₐₓ of about 32 dynes/cm to a film with a γₘₐₓ of about 70 dynes/cm (normal surfactant is represented by the lower-most tracing; data for surface films having 40, 50, 60, and 70 dynes/cm are represented by each of the progressively higher traces). These data demonstrate that increasing γₘₐₓ (from, e.g., about 32 to 70 dynes/cm), increases the ability of a surface film to support an increasing amount of distending pressure and thus protect a greater fraction of alveoli from potential fiber damage.

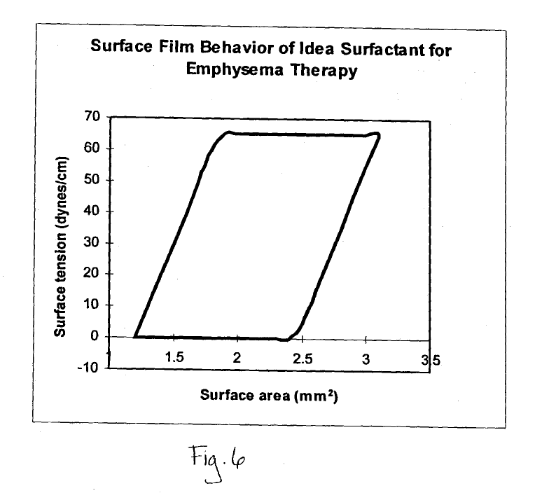

In general, a high value of γₘₐₓ, which is desirable for the purposes described here, is also associated with an elevated γₘᵢₙ. Unfortunately, such a film is not likely to be therapeutically useful, since a film must exert a minimum surface tension near zero to prevent alveolar collapse at end expiration. Accordingly, a surface film useful in treating a patient with lung disease (whose fiber network is stressed) will have biophysical properties similar to those depicted in FIG. 6: a high maximum surface tension at full surface film expansion and a low minimum surface tension at film compression. Thus, the compositions of the invention encompass lipid-based compositions that exert surface tensions substantially the same as those shown in FIG. 6 for alveolar surface areas from about 1.0 to about 3.0 mm². For example, a composition of the invention can exert a maximum surface tension of between about 60 and 70 dynes/cm (e.g., 55, 56, 57, 58, 59, 60, 61, 62, 63, 64, 65, 66, 67, 68, 69, 70, 71, or 72 dynes/cm) as it expands over alveoli whose surface area is increasing with inspiration and a minimum surface tension of between 0 and about 10 dynes/cm (e.g., 0, 1, 2, 3, 4, 5, 6, 7, 8, 9, 10, 11, 12, 13, 14, or 15 dynes/cm) as it compresses in alveoli whose surface area is decreasing with expiration. (The ascending and upper transverse arm of the graph represents the change in surface tension during inspiration and the descending and lower transverse arm of the graph represents the change during expiration).

As described in the Examples below, tissue-based and computer-based models have been used to analyze and define the biophysical properties of the surface films of the invention, and they can be used to readily test surface films having various components (including one or more of the components described herein) to determine whether those films have the requisite biophysical properties. Films that perform well in these models can be tested in animal models of pulmonary disease.

Useful surface films include those having a γ* (see Example 2) ranging from about 30 to about 70 dynes/cm (e.g., 30, 35, 40, 45, 50, 55, 60, 65, or 70 dynes/cm). Indeed, an important difference between a naturally occurring surfactant and surface films that can be used as biophysical stents to balance Pdistending (particularly in patients with emphysema) is γ*. γ* should be greater in the surface films than it is in naturally occurring surfactants. In addition, surface films useful in balancing P_{distending} can have one or more of the following biophysical characteristics: k₁ of about 6×10⁵ ml/g/min; k₂ of about 5 ml/g; and an m₂ of about 170 dynes/cm. Preferably, the surface films achieve dual objectives. First, they prevent the potential damaging effects of distending pressures on the interstitial fiber network in the lung. Second, and at the same time, they help stabilize alveoli at the end of an expiration, when they would be most susceptible to collapse.

The specific biophysical characteristics required of surface film can be endowed by a number of compositions that vary in the amount and type of lipids they contain. Specific combinations of lipids have been tested in the tissue-based, computer-based, and in vivo models described herein, and other combinations could readily be tested in these or similar models (e.g., the use of Brewster angle microscopy and atomic force microscopy).

Example 3 describes various surface films and Table 1, which summarizes the biophysical characteristics of a number of these, demonstrates that similar biophysical behavior can be generated using a variety of distinct lipid profiles.

TABLE 1

| Composition | k₁ | k₂ | γₘᵢₙ | γ* | m₂ |
|---|---|---|---|---|---|
| DAPC (0.7) + PG (0.2) + | 6 × 10⁵ | 6 | <0.5 | 38 | 170 |
| DPPC (0.05) + AA (0.05) | | | | | |
| DAPC (0.7) + DPPC (0.2) + | 6 × 10⁵ | 2 | <0.5 | 45 | 170 |
| AA (0.05) + PA (0.05) | | | | | |
| DPPC (0.7) + PG (0.2)+ | 3 × 10⁵ | 10 | <0.5 | 43 | 170 |
| AA (0.075) + Chol (0.025) | | | | | |
| DAPC (0.65) + PG (0.15) + | 6 × 10⁵ | 8 | <0.5 | 51 | 170 |
| AA (0.1) + PA (0.08) + | | | | | |
| synthetic SPC (0.02) | | | | | |

While the compositions presented here are mixtures comprised almost entirely of lipid components, naturally occurring proteins or synthetic peptides can also be included. In fact, inclusion of these proteins or peptides can also impart desirable biophysical properties on the compositions. More specifically analogs of native surfactant proteins and/or synthetic amphipathic short chain α-helical peptides, which have been shown to augment the function of synthetic lipid mixtures in vitro can be included (see, e.g., McLean et al., Am. Rev. Resp. Dis. 147:462-465,1993; Lipp et al., Science 273:1196-1199, 1996; Nilsson et al., Eur. J. Biochem. 255:116-124, 1998; and Gustafsson et al. FEBS Letters, 384:185-188, 1996).

The surface films described herein have specific benefits in the physiological context of emphysema and in vivo studies confirm the benefits suggested by ex vivo testing (see Example 4). These compositions specifically increase recoil at high lung volumes and promote a reduction in gas trapping, presumably by causing selective collapse of enlarged dysfunctional zones of lung.

As noted above, the compositions of the invention are useful in treating patients who have emphysema, including those patients who have undergone a lung volume reduction procedure. Mechanical forces, which are important in the progression of emphysema, are pronounced following lung volume reduction, when damaged lung tissue is stretched in an attempt to make it function better. The re-stretching process, however, increases the tension in the tissue and promotes ongoing tissue fiber failure. This is manifest clinically as a rapid decline in lung function.

Formulations and Use

The compositions of the present invention can be formulated as dry powders, and they can be reconstituted before use. For example, a surface film having biophysical characteristics appropriate for treating emphysema can be formulated as a dry powder and reconstituted with water (e.g., sterile, preservative-free water) prior to administration. When possible, and whenever preservatives or anti-microbial agents are omitted, the surface films should be reconstituted using an aseptic technique. The reconstituted surface films are expected to remain sterile and stable for about 24 hours if stored between about 2 and 8° C. When aseptic technique cannot be ensured, reconstitution should preferably take place immediately before use and any unused suspension should be discarded.

In the event a patient is unconscious and intubated, the total dose can be administered by way of the endotrachael tube. The rate of administration can be varied and should be sufficient to allow the reconstituted suspension to pass through the tube (or a device, such as a catheter inserted within the tube) and into the lungs without accumulation. The studies conducted to date indicate that the minimum recommended time for administration of the full dose will be about four minutes. Dosing should be slowed or interrupted if the patient's condition deteriorates. Signs and symptoms of deterioration include a loss of skin color (patient appears pale or ashen), slowing or irregular heart rate, and more than a transient depression of arterial oxygen concentration. Dosing should also be slowed or interrupted if the surface film accumulates in the endotracheal tube.

The surface films can be supplied in the form of a kit that, in addition to the surface film, contains, for example, a vial of sterile water, physiologically acceptable buffer, or other physiologically acceptable suspension medium, carrier, or diluent. Optionally, the kit can contain an atomizer system to generate particulate matter (atomizers are presently commercially available) and instructions for use (which may be printed, on audio or videocassette, or both) and other material describing, for example, possible side effects.

Other methods of administration are suitable, and they include all those presently considered appropriate and effective for replacement surfactant therapy. A direct and effective method is instillation of the surface film into the lung through the trachea. The film can be administered as a liquid solution in water or buffered physiological solutions (e.g., saline, PBS, or the like), and can be administered over a period of several minutes (e.g., 5-15 (e.g., about 6, 8, 10, 12, or 14 minutes). The studies conducted to date indicate that typical dosages can range from about 10 to about 300 milligrams of surface film per kilogram of patient body weight, and are preferably from about 25 to about 125 mg/kg (e.g., 25, 30, 35, 40, 45, 50, 75, or 100 mg/kg). The surface film can be administered hourly, once or several times in a day (e.g, every 4, 6, 8, 12, or 24 hours), several times in one week, regularly over time (e.g., weekly, bi-weekly, monthly, or semi-annually), or irregularly on an as-needed basis.

A useful mechanism for delivery of the powder into the lungs of a patient is through a portable inhaler device suitable for dry powder inhalation. Many such devices, typically designed to deliver anti-asthmatic agents (e.g., bronchodilators and steroids) or anti-inflammatory agents into the respiratory system are commercially available. The device can be a dry powder inhaler, which can be designed to protect the powder from moisture and to minimize any risk from occasional large doses. In addition, the device can protect the surface film from light and can provide one or more of the following: a high respirable fraction and high lung deposition in a broad flow rate interval; low deviation of dose and respirable fraction; low retention of powder in the mouthpiece; low adsorption to the inhaler surfaces; flexibility in dose size; and low inhalation resistance. The inhaler can be a single-dose inhaler or a multi-dose inhaler.

The surface film, in powder form, can be manufactured in several ways, using conventional techniques. One can, if desired, micronize the active compounds (e.g., one or more of the lipids). One can also use a suitable mill (e.g., a jet mill) to produce primary particles in a size range appropriate for maximal deposition in the lower respiratory tract (i.e., under 10 μM). For example, one can dry mix lipids and other components of the surface film (e.g., proteins or peptides) and a carrier (where appropriate) and micronize the substances together. Alternatively, the substances can be micronized separately and then mixed. Where the compounds to be mixed have different physical properties (e.g., hardness or brittleness), resistance to micronization varies, and each compound may require a different pressure to be broken down to suitable particle sizes

It is also possible to dissolve the components first in a suitable solvent (e.g., sterile water, PBS, or the like) to obtain mixing on the molecular level. When this is done, one can adjust the pH value to a desired level. To obtain a powder, the solvent should be removed by a process that allows the components of the surface film to retain their biological activity. Suitable drying methods include vacuum concentration, open drying, spray drying, and freeze-drying. After being dried, the solid material can, if necessary, be ground to obtain a coarse powder, and further, if necessary, micronized.

In addition, and if desired, the micronized powder can be processed to improve the way in which it flows through and out of inhaler (or other) devices. For example, the powder can be processed by dry granulation to form spherical agglomerates with superior handling characteristics. In that case, the device would be configured to ensure that no substantial agglomerates exit the device. A possible advantage of this process is that the particles entering the respiratory tract of the patient are largely within the desired size range.

The delivery apparatus can also be a nebulizer that generates an aerosol cloud containing the components of the surface film. Nebulizers are known in the art and can be a jet nebulizer (air or liquid; see, e.g., EP-A-0627266 and WO 94/07607), an ultrasonic nebulizer, or a pressure mesh nebulizer. Ultrasonic nebulizers, which nebulize a liquid using ultrasonic waves usually developed with an oscillating piezoelectric element, take many forms (see, e.g., U.S. Pat. Nos. 4,533,082 and 5,261,601, and WO 97/29851). Pressure mesh nebulizers, which may or may not include a piezoelectric element, are disclosed in WO 96/13292.

Nebulizers, together with dry powder and metered dose inhalers, are commonly used to deliver substances to the pulmonary air passages. Metered dose inhalers are popular, and they may be used to deliver medicaments in a solubilized form or as a dispersion (the propellant system historically included one or more chlorofluorocarbons, but these are being replaced with environmentally friendly propellants). Typically, these inhalers include a relatively high vapor pressure propellant that forces aerosolized medication into the respiratory tract upon activation of the device. To the contrary, dry powder inhalers generally rely entirely on patients' inspiratory efforts to introduce a medicament in a dry powder form to the lungs. Nebulizers form a medicament aerosol by imparting energy to a liquid solution. More recently, therapeutic agents have been delivered to the lungs during liquid ventilation or pulmonary lavage using a fluorochemical medium.

### EXAMPLES

### Example 1

A Tissue-Based Model of Emphysema.

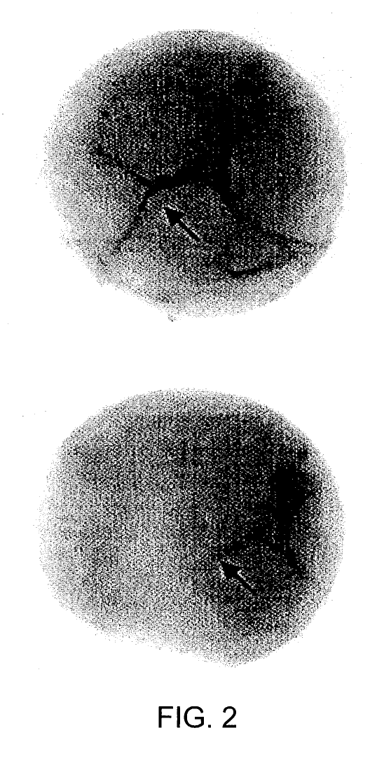

The collagen and elastin fibers within the walls of the alveoli can be visualized and otherwise examined in a number of circumstances (see, e.g., FIG. 2). For example, lung tissue containing alveoli can be obtained from healthy animals (including human patients) or from humans or other mammals that have enlarged alveoli as the result of a natural or experimentally induced disease process, such as emphysema. The tissue can be mechanically stretched with a force that mimics the force the tissue is subjected to in vivo during breathing (including shallow, normal, or deep breathing), and it can be stretched in the presence or absence of pharmaceutical compositions, such as known surfactants or the surface films of the present invention to assess the ability of those compositions to reduce fiber breakage.

As noted above, when alveoli are enlarged, fiber breakage occurs at strains that approximate those of normal breathing. This effect can occur on a global scale throughout the lung, but is more likely to occur on a regional scale, at a specific locus. But in either event, it can cause rapid and self-propagating progression of tissue damage due to stress-related rupture of tissue network fibers, and that rupture contributes to the progression of emphysema.

The stress-strain relationships of tissue strips isolated from rats with experimental emphysema were characterized. Collagen and elastin fibers were directly visualized using fluorescent antibody labeling during application of mechanical stress in an organ bath system to examine the geometry and integrity of the fiber network during cyclic stress. One of the resulting microscopy images is shown in FIG. 2. With increasing stretch, fibers become more distorted. At strains that approximate those of normal breathing, fiber breakage was observed. This is analogous to what happens in patients with severe end stage emphysema once they reach a critical level of tissue destruction.

### Example 2

A Computer-Based Model of Emphysema, with Implications for Lung Volume Reduction.

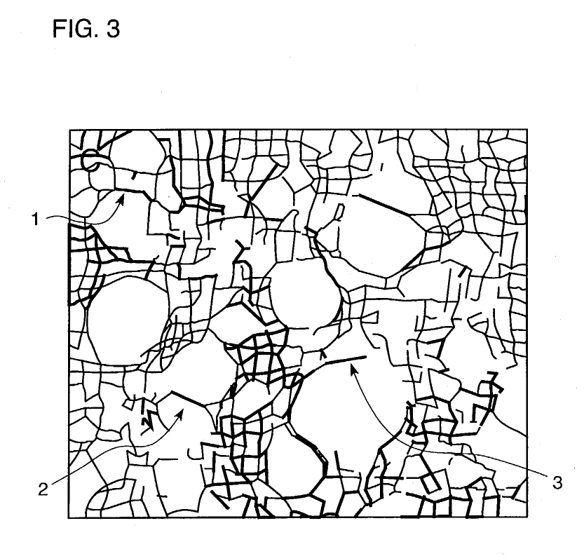

A finite element computer model was used to simulate a lung composed of a network of stress-supporting fibers equivalent to the collagen and elastin fibers in the alveolar wall. Utilizing parameter values that are representative of human lung physiology, this model identifies foci of high stress concentrations, which tend to localize along the edges of small bullae. Under stretch, fibers under high tensile stress (shown in FIG. 3 and labeled as fibers 1, 2, and 3) undergo rupture, which leads to enlargement of the bullae and amplification of regional stress concentrations. This process becomes self-propagating as rupture leads to further weakening. The net result is equivalent to what is seen in clinical practice and is consistent with observations made following LVRS. Despite initial improvement, there is an eventual and rapid decline in lung function following LVRS. The procedure is performed to increase tissue recoil, but this can simultaneously cause an increase in the stress field within the fiber network.

One might expect stress-related changes to have a pronounced impact on lung physiology in patients who have undergone a lung volume reduction procedure because these patients generally have severe lung disease and significant tissue destruction. But the procedure is, obviously, an external perturbation and it imposes a sudden “step change” in fiber-borne stresses because it increases elastic recoil pressures. (Although patients with emphysema have fewer fibers to support the stress of breathing, overall stress is reduced as a consequence of a stress relaxation process that is part of the natural history of emphysema). While lung volume reduction has beneficial effects on lung physiology in the short term, it can also cause an accelerated rate of fiber rupture in the longer term according to the mechanism simulated in computer models and observed in the tissue strip experiments described in Example 1.

As noted above, recoil pressure is generated by at least two components, a “tissue” component generated by the fiber network and a “surface tension” component generated by the surface film according to the equation:

P_{recoil}=Pₜᵢₛₛᵤₑ+P_{surface tension}.

LVRS increases recoil pressure by increasing Pₜᵢₛₛᵤₑ, which causes damage to the fiber network; surface film therapy increases recoil pressure by increasing P_{surface tension}, which does not damage the fiber network.

This Example demonstrates that computer models can be used to evaluate stress on fibers within the lung in any of a number of circumstances. They can be used, for example, to simulate lung tissue in healthy animals (including human patients) or in animals that have enlarged alveoli, as occurs in emphysema, under a variety of conditions (e.g., shallow, normal, or deep breathing). They can also be used to simulate lung tissue after lung volume has been reduced (by a surgical or non-surgical lung volume reduction procedure) and to simulate tissue that has been treated with a known surfactant, surfactant replacement, or a surface film of the present invention. One can, therefore, use computer models, such as that described here, to assess the ability of those compositions to reduce fiber breakage.

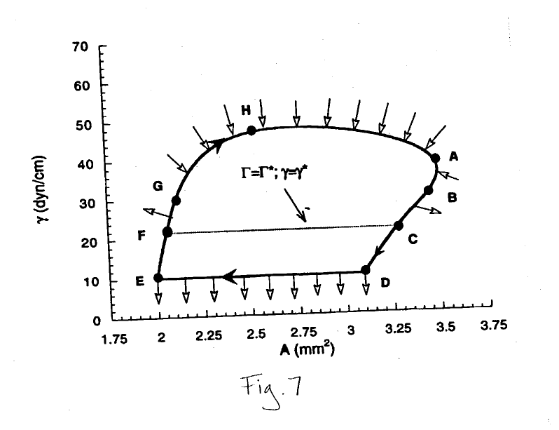

A computer model based on first principles has been used to characterize the interfacial behavior of surface films from surface tension-surface area profiles measured using a surface balance device (Ingenito et al. Appl Physiol. 86:1702-1714, 1999). The model used in this example assumes that dynamic interfacial behavior can be described in terms of three distinct processes, each of which applies at different times during cycling, depending upon whether the film is expanded (in a liquid state) or compressed (in a gel or solid phase; see FIG. 7). A computer model can characterize surfactant (or any surface film) transport to and from the interface in terms of three distinct surface concentration regimes.

In the first regime, the surface concentration (Γ, measured in moles of surfactant per cm² surface area) is less than the maximum equilibrium surface concentration (Γ*) that can be achieved as bulk phase concentration (C) is increased. This is represented by segment FC on FIG. 7. In this regime, adsorption and desorption to and from the interface are assumed to occur according to the Langmuir relationship:

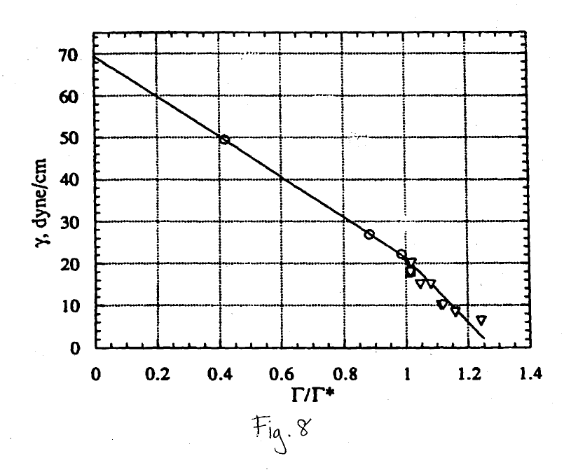

dM/dt=A{k₁C(Γ*−Γ)−k₂Γ}

where t is time, k₁ is the adsorption coefficient, k₂ is the desorption coefficient, A is the interfacial area, and M=ΓA the amount of surfactant (or surface film) in the interface. Surface tension (γ) is related to surface concentration through the static isotherm relationship, which is shown to decrease linearly with increasing surface concentration Γ such that γ=70 dynes/cm when Γ/Γ*=0, and γ=γ* when Γ/Γ*=1. This relationship defines the isotherm slope m₁=−dγ/d(Γ/Γ*). See FIG. 8.

In the second regime, shown in FIG. 7 as segments CD and EF, surface concentration Γ is greater than Γ*. However, Γ remains less than the maximum concentration (Γₘₐₓ) that can be achieved during lateral compression of surface active material at the interface. In this regime, the surfactant (or surface film) is modeled as insoluble, meaning it does not exchange surface active material with the bulk phase. As shown in FIG. 8, the relationship between γ and Γ/Γ* in this regime decreases linearly with a slope, −m₂, that is distinct from m₁. It is important to note that this region cannot be characterized from static measurements of surfactant. The film must undergo external dynamic compression to reach these low surface tensions.

In the third regime, shown in FIG. 7 as segment DE, Γ is equal to Γₘₐₓ. Surfactant molecules are packed as tightly as possible in the interface, and surface concentration cannot increase further. Surface tension reaches its minimum value (γₘᵢₙ) at this point and remains constant as surface area is further decreased by film compression. Any further compression leads to material being lost from the surface to the bulk by squeeze-out or film collapse.

γ* is defined as the lowest equilibrium surface tension measured as bulk concentration was increased up to 5 mg/ml; it corresponds to a surface concentration of surfactant equal to Γ*. The lowest surface tension achieved during dynamic film compression at the highest bulk concentration (1 mg/ml) studied determines γₘᵢₙ. The isotherm slope m₂ was determined using the surface tension versus surface area slope (dγ/dA) in the insoluble regime during dynamic oscillations (segment CD of FIG. 7) as surface tension was decreased from γ* to γₘᵢₙ during film compression for samples at high bulk concentration (1 mg/ml). m2 is defined as the slope dγ/d(Γ/Γ*) when Γ/Γ* is >1. This slope is determined experimentally during quasi-static film compression by measuring surface tension, and assuming that once surface tension begins to decrease, the amount of surfactant material within the surface film remains constant. Thus, surface concentration, and surface tension change solely as a consequence of changes in surface area rather than changes in the number of surfactant molecules at the air-liquid interface.

Estimation of model parameters for describing surface film biophysics. Model behavior is determined by five parameters: the surfactant adsorption (k₁) and desorption (k₂) rate constants in regime (i), the minimum equilibrium surface tension (γ*), the slope m₂, and the minimum achievable surface tension during film compression (γₘᵢₙ). Note that m₁ is determined by γ*. These parameters can be estimated from equilibrium and dynamic surface tension measurements made in vitro using a device such as the pulsating bubble surfactometer.

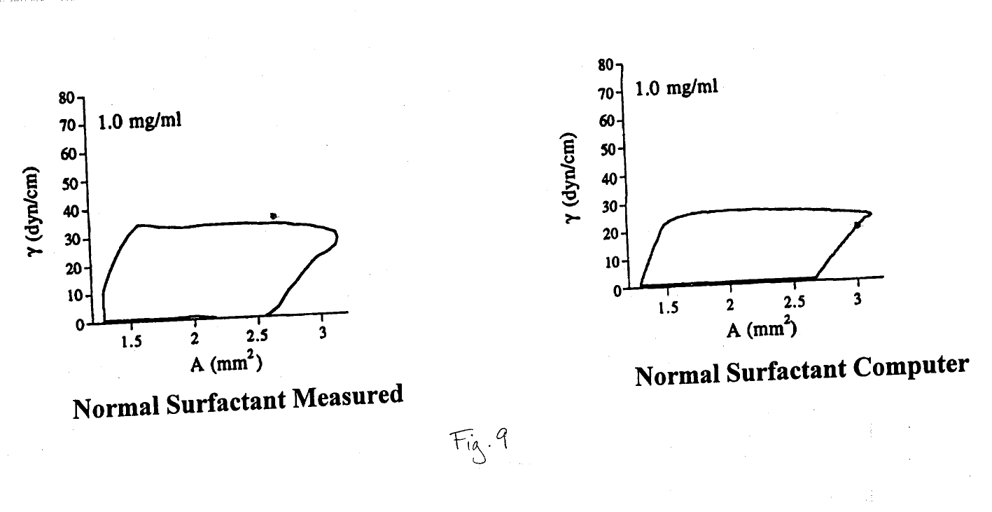

By describing surface film behavior in terms of this parameter set, it is possible to readily compare, and fully characterize, the biophysical properties of surface films with any specified biophysical profile. Referring to FIG. 9, the left hand panel shows the surface tension-surface area profile measured for normal calf lung surfactant, while the right hand panel shows a corresponding “matching” computer simulation using the following parameter set: k₁=6×10⁵ ml/g/min; k₂=5 ml/g; γ*=22.2 dynes/cm; γmin<0.5 dynes/cm; and slope B to D (designated m₂)=170 dynes/cm. As shown in FIG. 9, simulations performed using this parameter set are nearly identical to those measured using the pulsating surfactometer. Using the model, it is possible to determine what combinations of parameters are required to generate a surface film with biophysical properties similar to those represented by the surface tension surface area profile shown in FIG. 6 (surface films having such a profile being within the scope of the present invention and useful in treating patients with lung diseases such as emphysema). Simulations were performed by systematically varying each of the parameters in the computer model over a range of values until a combination was determined that matched the desired target profiles. While this approach does not guarantee that the set of biophysical parameters, or the specific composition of the film describing the surface tension-surface area profile deemed desirable, is unique, uniqueness is not essential for development of a useful product. Any combination of lipids, or lipids and proteins and/or polysaccharides that maintains surface tensions below 5 dynes/cm during film compression and achieves surface tensions greater than 50 dynes/cm could serve the desired purpose (e.g., could serve as an effective treatment of patients with emphysema).

The parameter set that best matched the behavior of the hypothetical “ideal” surface film for supporting the fiber network in emphysema is the following: k₁=6×10⁵ ml/g/min; k₂=5 ml/g; m₂=170 dynes/cm; and γ* ranging from about 20 to about 70 dynes/cm (e.g 30-65 dynes/cm). Perhaps the most important parameter change required to produce an alteration in film behavior from normal surfactant to the hypothetical ideal that can be used as a “biophysical stent” is an increase in γ*.

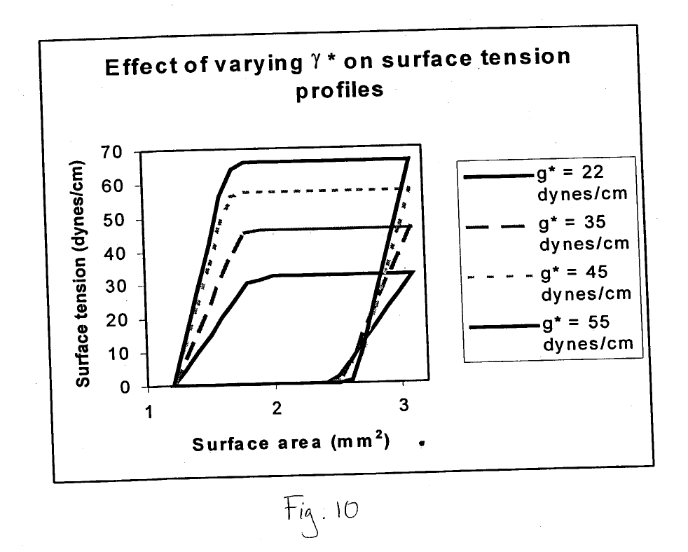

Simulations depicting how surface tension versus surface area changes with systematic increases in γ* are shown in FIG. 10. These simulations confirm that a lipid combination with the ability to absorb rapidly, sustain high surface pressures during dynamic compression, and have an equilibrium surface tension of >40 dynes/cm is useful. Such a film can accomplish the dual objectives of preventing the potential damaging effects of distending pressures on the interstitial fiber network in the lung, while simultaneously stabilizing alveoli at end expiration when they would be most subject to collapse.

Under static conditions, surfactant films that adsorb to an air-liquid interface display the unique property that surface tension varies with the geometric dimensions of the structure upon which the film has spread in accordance with Laplace's law. The modeling analysis presented above further indicates that during dynamic cycling, surface tension is a function of the amplitude of variation of the characteristic dimension of the system. Specifically, this means that surface tension varies as a function of ΔA/A, the amplitude of surface area change relative to the magnitude of the area itself. This behavior characteristic relates specifically to the biophysics of Langmuir kinetics. When surface films undergo large excursions relative to the mean subtended area, more surface-active material moves into the interface than during small excursions. As a result, following a large relative area excursion, films undergoing compression are more readily able to reach low surface tensions than films following a small area excursion.

These unique static and dynamic biophysical properties have important implications with respect to the potential utility of administering surface films to patients with emphysema with the specific objective of utilizing them as a biochemical stent. Altering the surface film in such a manner as to increase γ_{equil} and γₘₐₓ would tend to increase recoil, resulting in a new equilibrium at a smaller bubble size. These same films would, however, not have a detrimental effect on the more normal areas of lung where ΔA/A is larger, since the greater excursions would tend to generate lower surface tensions, and impart mechanical stability. Therefore, surface films that satisfy these biophysical characteristics have the potential of benefiting patients with emphysema both before and after volume reduction therapy through two distinct mechanisms. First, independent of alveolar size, surface films can provide mechanical support to the parenchymal fiber network by generating high surface tension and large P_{surface film}, imparting a larger recoil to the alveolar septae during lung inflation than a normal surfactant film. This would reduce the stress on the collagen and elastin components of the individual fibers within the network, and reduce the tendency for fiber rupture. Second, for the largest alveoli, which represent those located in the most damaged regions of lung, films with these properties preferentially impart a greater static recoil, a greater tendency for collapse, and a greater tendency to cause chemical “lung volume reduction,” than to other less affected regions.

### Example 3

As noted above, in diseases where surfactant dysfunction is the primary abnormality, researchers and physicians aim to supply surfactant replacements that have characteristics of normal surfactants. The objective is to lower surface tension and restore alveolar stability by administering surfactants with these biophysical properties (as defined by our computer model system): k₁=6×105 ml/g/min; k₂=5 ml/g; γ*=22.2 dynes/cm; γₘᵢₙ <0.5 dynes/cm; and slope B to D (designated m₂)=170 dynes/cm. Again, as noted above, although such a composition would be an effective surfactant replacement, it would not be an effective therapeutic agent for treating emphysema.

We undertook a systematic analysis of the biophysical properties of a wide range of lipid-based replacement therapies to assess which lipid combinations might provide γ* values of between 35-65, while providing k₁, k₂, γₘᵢₙ, and m₂ values similar to those of native surfactant.

Candidate lipid samples were prepared in normal saline containing 1.5 mM CaCl₂, sonicated using a microprobe sonicator 3×20 seconds on ice, and then loaded into a pulsating bubble surfactometer for determination of surface tension versus surface area profiles at 1, 20, and 100 cycles/min as previously described (Ingenito et al., J. Appl. Physiol. 86:1702-1714, 1999). Samples were measured at concentrations of 1.0, 0.1, and 0.01 mg/ml to allow for complete characterization of biophysical properties. Measured profiles at each concentration and cycling frequency were then matched to computer simulations as previously described by Otis et al. (J. Appl. Physiol. 77:2681-2688, 1994) to provide estimates of k₁, k₂, m₂, γ*, and γₘᵢₙ values. Using this approach, we identified a unique combination of lipids with biophysical properties that match the specific design characteristics outlined above for a film that would be capable of imparting protection to the fiber network of the lung, particularly in areas affected by emphysema.

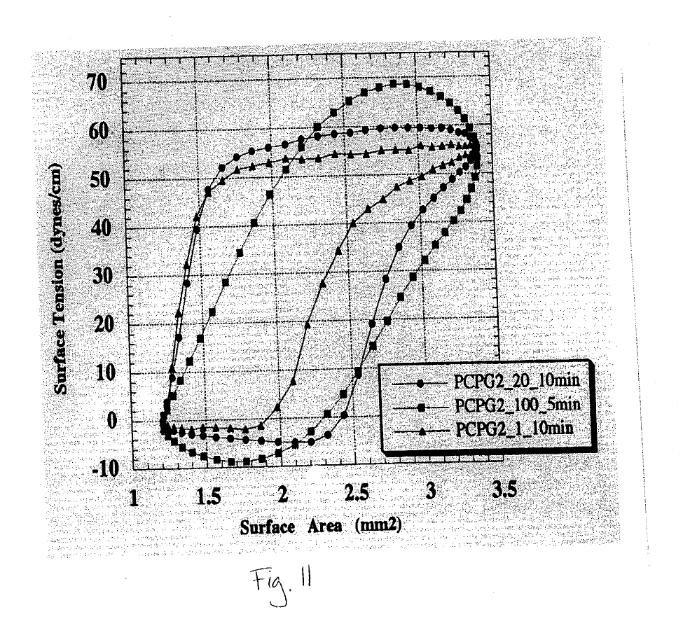

In one configuration, the lipid mixture consists of 70% di-arachidoyl-phosphatidylcholine (PC), 25% phosphatidylglycerol (PG), 2.5% palmitic acid (PA), and 2.5% arachidic acid (AA). Representative surface tension-surface area profiles for a dA/A of 75% are shown in FIG. 11.

This combination of phospholipids and fatty acids is biocompatible, synthetic, and non-immunogenic. The individual reagents are inexpensive to purchase and reconstitute, and can be easily administered via a nebulizer, or prepared as a dry powder for turbohaler administration.

Several other compositions we have tested have characteristics that, while perhaps not as desirable as the composition just described, could nevertheless be used in emphysema treatment. These compositions include dialmitoylphosphatidylcholine combined with phosphatidylglycerol and palmitic acid as a 65:25:10% mixture; di-palmitoylphosphatidylcholine combined with phosphatidylglycerol in a 70:30% mixture; and di-arachidoylphosphatidylcholine and palmitoylphosphatidylcholine combined together such that the two add up to 70% of the total mixture, with the additional 30% composed of phosphatidylglycerol with or without up to 10% fatty acids including arachidic acid or palmitic acid and several percent cholesterol.

### Example 4

A variety of small animal models with specific characteristics of human emphysema have been developed and utilized in clinical research. Each has specific characteristics that make it suited for addressing one or more questions relating to this disease. For the purposes of this work, a model displaying physiological characteristics of hyperexpansion and loss of elastic recoil pressure is needed to test the hypothesis that administration of this mixture could increase recoil pressure without causing marked abnormalities in gas exchange due to alveolar collapse and shunt propagation.

Several strains of genetically altered mice that display these essential physiological properties have been engineered and characterized (Shapiro et al., Am J Respir Cell Mol Biol. 22:4-7, 2000). These include the Tightskin mouse (Tsk +/−), Blotchy mouse (Blo), SP-D knockout mice, Collagenase transgenic mouse, klotho transgenic mouse, IL-11 transgenic mouse, and PDGF-A knockout mouse. Some strains are commercially available from Jackson Laboratories (Bar Harbor, Me.). Tsk mice were used in this initial study, and physiology was compared to that of wildtype C57BL/6 mice.

Mice were maintained in a virus-free facility and were studied 1 and 3 weeks following delivery from the supplier (6-8 weeks of age). Twelve Tsk (+/−) mice (weight 19.6±3.7 g) and twelve C57BL/6 mice (weight 21.3±1.6 g) (Jackson Laboratories) were each divided into two groups. Group I animals (n=6 for both strains) served as controls, and had baseline static and dynamic lung function measured, as well as repeat measurements following administration of saline alone. Group II animals (n=6 for both strains) comprised the test group, and similarly had baseline static and dynamic lung function measured, as well as repeat values determined after administration of the nebulized, sonicated lipid test mixture.

Animals were anesthetized with intra-peritoneal pentobarbital (60 mg/kg), and had a tracheal cannula placed. A small subxiphoid incision was made to expose the intrathoracic cavity and allowing for the determination of transpulmonary pressure from assessment of mouth pressure referenced to atmospheric pressure. Prior to initiating mechanical ventilation, animal lungs were inflated once to 0.75 ml to ensure that all measurements reflected a similar volume history, or pressure-volume state for the lung. Ventilator support was administered at settings of 0.3 ml tidal volume, 150 breaths/min, Fio₂=0.21 (room air) with 3 cm H₂O positive end-expiratory pressure using a computer controlled volume cycled small animal ventilator.

Dynamic measurements of lung function were performed using the optimal ventilator waveform (OVW) method of Lutchen et al. (J. Appl. Physiol. 75:478-488, 1993). A forced oscillatory volume waveform, with energy at multiple frequencies, is applied as an input signal, and trans-pulmonary pressure is measured as the dependent output variable. Forcing frequencies and amplitudes are selected to provide effective tidal ventilation, while simultaneously allowing assessment of lung impedance over a range of frequencies. Low frequency responses provide specific information about the tissue component of lung resistance (Rti), high frequency responses allow for accurate assessment of airway resistance (Raw), and the pattern of change in elastance (EL) with frequency provides information about heterogeneity of time constants within the lung. Measurements of lung mechanics were performed in triplicate, and lung resistance and dynamic elastance were expressed as functions of frequency. Results were summarized by fitting the impedance data to the constant phase model of Hantos et al (J. Appl. Physiol. 73:427-433, 1992) which describes the viscoelastic properties of the lung:

P(ω)/V(ω)=Raw+G/ω^{α}−jH/ω^{1-α}

By using this approach, it is possible to summarize dynamic lung physiology in terms of three parameters: Raw, G (which describes tissue resistance), and H (which describes tissue elastance).

To assess the effects of each inhalation therapy on lung mechanics, physiological measurements were recorded in triplicate prior to exposure (baseline) and at 2, 10, 20, and 60 minutes following exposure. Quasi-static inflation-deflation curves were recorded from Ptp=0 to Ptp=25 cm H₂O at baseline, 10 minutes post-inhalation, and 60 minutes post-inhalation.

Following the completion of each experiment, the heart and lungs were removed en bloc, and the heart and excess mediastinal tissues were dissected free. The trachea was tied off at a Ptp of 0, and absolute lung volume was measured by volume displacement in a calibrated 10 ml graduated cylinder. This volume was then used to construct a quasi-static pressure volume curve referenced, not to an individual animals lung volume at Ptp=0 (since this might differ from animal to animal), but rather to an absolute measured lung volume.

Quasi-static pressure volume relationships were then fit to the exponential relationship of Salazaar and Knowles (J. Appl. Physiol. 19:97-104, 1964) such that responses could be characterized quantitatively in terms of specific physiological parameters. The relationship used was:

V(P)=Vₘₐₓ−Ae−^{kP}

where Vₘₐₓ is the lung volume approached at infinite pressure, A=Vₘₐₓ−Vₘᵢₙ, Vₘᵢₙ is the lung volume at 0 distending pressure, k is the shape factor which describes the profile of the fit between pressure and volume, V is volume, and P is transpulmonary pressure. Using this expression, the pressure-volume relationship can be uniquely described in terms of Vₘₐₓ, Vₘᵢₙ, and k.

Changes in physiology within each group resulting from inhalation therapy were assessed for statistical significance using ANOVA for repeated measures. Changes in physiology between groups were assessed by two way ANOVA. Statistical significance was defined as p<0.05.

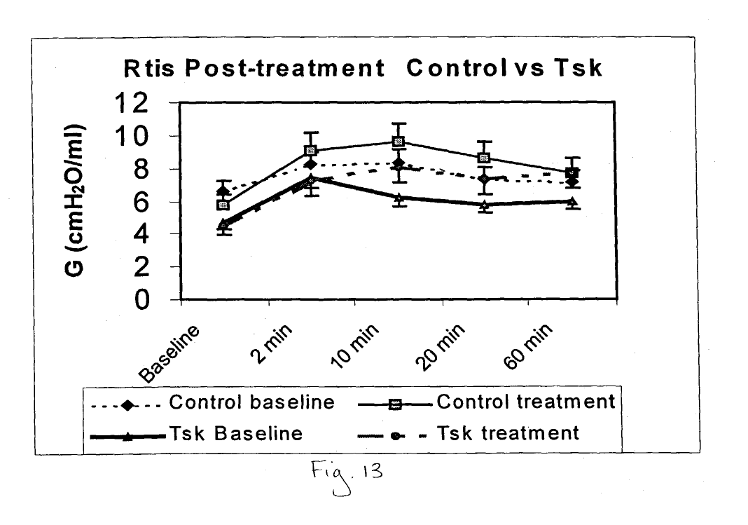

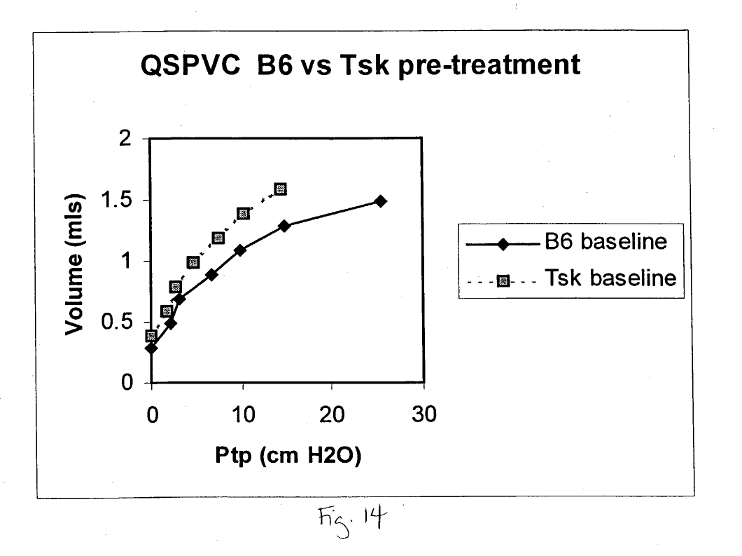

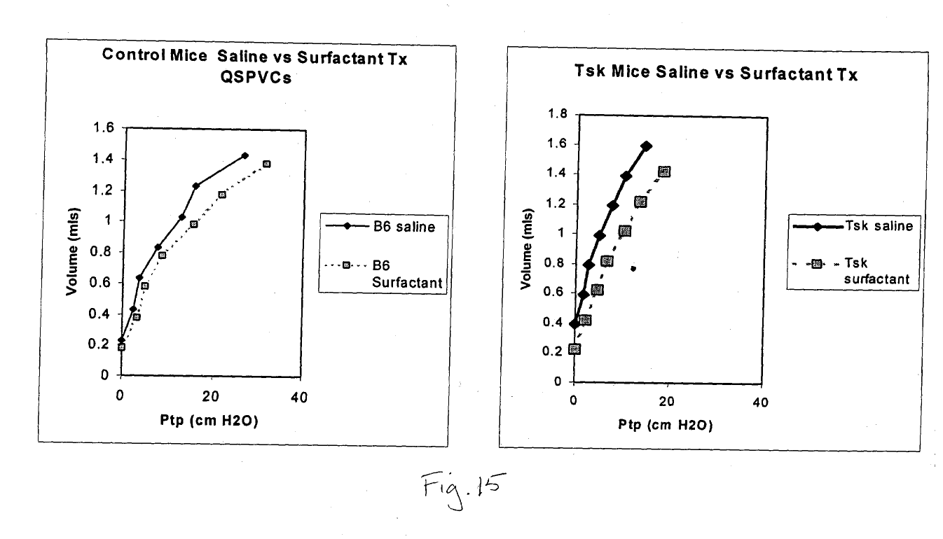

Results of lung physiology measurements pre- and post-saline and surface film inhalation are summarized in FIGS. 13 through 17. Airway resistance increased in B6 control mice following administration of a surface film, possibly due to an effect on small airways. In Tsk mice, the effect of surface film administration on airway physiology was minimal. Surface film administration had a more pronounced effect on lung tissue mechanics than airway physiology (as shown in FIGS. 14 and 15). Surface film administration caused a sustained, statistically significant increase in tissue resistance in B6 (5.75±0.71 at time 0 vs 7.70±0.82 cm H₂O/ml time 60 minutes, 34% increase, p<0.05 by paired t test) and Tsk (4.51±0.66 vs 7.73±0.92 cm H₂O/ml, 71% increase, p<0.05 by paired t test) mice. Surface film administration caused similar changes in dynamic elastance values (FIG. 15) in both strains. Among B6 mice, elastance increased 55% (28.2±4.6 vs 43.5±7.8 cm H₂O/ml, p<0.05 by paired t test) following treatment, while Tsk mice experienced a 56% increase (21.0±5.2 vs 32.7±6.9 cm H₂O/ml, p<0.05 by paired t test). These results demonstrate that this surface film is capable of producing lasting dynamic physiological effects of the type expected to be beneficial in human patients with emphysema.

Static lung physiology, summarized in FIGS. 16 and 17, shows similar desirable physiological effects. FIG. 16 depicts baseline static lung mechanics in B6 control and Tsk emphysema mice. Recoil pressures at all volumes are diminished in Tsk mice, and retained gas volume at 0 Ptp was greater among Tsk mice than B6 mice. These findings are consistent with the physiology of emphysema, and suggest that the pathological changes observed among Tsk animals do, in fact, correspond with abnormal physiology.

The effect of surfactant inhalation on static lung mechanics is summarized in FIG. 17. QSPVCs 60 minutes after saline inhalation are compared to those measured 60 minutes following surface film inhalation in both strains of mice. Administration of the therapeutic composition caused recoil pressures to increase at all lung volumes in both B6 and Tsk mice. Recoil pressures at total lung capacity (defined as the volume corresponding to 1.2 mls above that at 0 Ptp) increased similarly in both of these strains (26.5% in B6 mice, and 36% in Tsk mice). Surfactant therapy also reduced trapped gas volume in both strains. In B6 mice, lung volume at Ptp=0 was reduced 18%, while in Tsk mice this reduction was 44%.

QSPVC data was fit to the exponential data of Salazaar and Knowles to provide further insight into how surface films affect static lung mechanics. The results are summarized in Table 2, below. The therapeutic compositions caused a consistent reduction in the “shape factor” (parameter k), which determines the “curvature” of the exponential relationship between pressure and volume. This reduction means that treatment specifically causes recoil at higher lung volumes to be greater than following saline therapy in both B6 and Tsk mice, but indicates less of an effect at lower lung volumes (i.e., a volume specific recoil effect as suggested by in vitro surface tension surface area profiles. Treatment also produced a consistent reduction in gas trapping. This was reflected by a drop in Vₘᵢₙ, a reduction that was substantially larger in Tsk emphysema mice than in B6 control mice.

TABLE 2

| Parameter | B6 Sal | B6 surf | Tsk Sal | Tsk surf |
|---|---|---|---|---|
| k | 0.08 | 0.055 | 0.095 | 0.072 |
| Vmax (ml) | 1.59 | 1.65 | 2.02 | 1.86 |
| Vmin (ml) | 0.23 | 0.18 | 0.39 | 0.19 |

A number of embodiments of the invention have been described. Nevertheless, it will be understood that various modifications may be made without departing from the spirit and scope of the invention. For example, and as noted above, the lipid-containing compositions described herein can vary, and they can contain other biologically active or inactive components (e.g., proteins, peptides, polyethylene glycol, or other synthetic detergent formulations) so long as the compositions behave in a manner that allows them to increase maximum surface tension during film expansion and maintain a minimum surface tension <5 dynes/cm.

What is claimed is:

1. A pharmaceutically acceptable composition comprising a lipid, wherein the composition, when applied to an enlarged alveolus, exerts a surface tension within the alveolus that substantially reduces the stress on fibers within the alveolus when inflated by a normal inspiration.

2. The composition of claim 1, wherein the composition displays a surface tension-surface area profile in which surface tensions are large enough at the end of an inspiration to substantially reduce the stress on fibers within the alveolus and small enough at the end of an expiration to substantially prevent alveolar collapse.

3. The composition of claim 1, wherein the composition displays a surface tension-surface area profile substantially similar to the surface tension-surface area profile shown in FIG. 6.

4. The composition of claim 1, wherein the composition displays a γ* of about 30 to about 70 dynes/cm.

5. The composition of claim 4, wherein the composition displays a γ* of about 35 to about 60 dynes/cm.

6. The composition of claim 4, wherein the composition displays a γ* of about 45 to about 55 dynes/cm.

7. The composition of claim 4, wherein the composition displays a γ* of at least 55 dynes/cm.

8. The composition of claim 1, wherein the composition is formulated for administration by inhalation.

9. The composition of claim 1, wherein the composition comprises di-arachidonylphosphatidylcholine (DAPC).

10. The composition of claim 9, wherein the composition comprises at least 50% DAPC.

11. The composition of claim 9, further comprising di-palymitoylphosphatidylcholine (DPPC).

12. The composition of claim 9 or claim 11, further comprising phosphatidylglycerol.

13. The composition of claim 9 or claim 11, further comprising arachidic acid.

14. The composition of claim 9 or claim 11, further comprising cholesterol.

15. The composition of claim 1, wherein the composition comprises 50-80% di-arachidoylphosphatidylcholine (DAPC), 10-30% phosphatidylglycerol, 1-10% palmitic acid, and 1-10% arachidic acid, provided the total lipid composition does not exceed 100% of the composition.

16. The composition of claim 9 or claim 11, further comprising natural surfactant protein B, natural surfactant protein A, natural surfactant protein C, recombinant surfactant protein C, small alpha-helical peptides with hydrophobic characteristics, or peptide-like compounds.

17. The composition of claim 9 or claim 11, further comprising an anti-inflammatory agent, a steroid, a bronchodilator, an anti-cholinergic compound, or an agent that modulates inflammation or airway tone.

18. The composition of claim 17, wherein the steroid is hydrocortisone, dexamethasone, beclamethasone, or fluticasone.

19. A method of treating a patient who has emphysema or another pulmonary disease in which fibers within the alveoli are under stress, the method comprising to the patient the composition of claim 15.

20. The method of claim 19, wherein the patient is a human.

21. The method of claim 19, wherein the patient has undergone lung volume reduction therapy.
